SEQUENCE LISTING (1 sequence)

SEQ ID NO: 1 - protein, 207 aa, Homo sapiens
  SIGNAL (1)..(23)
  mat_peptide (24)..(207)
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

(19) United States
(12) Patent Application Publication
(10) Pub. No.: US 2010/0159479 A1
(43) Pub. Date: Jun. 24, 2010

(54) TIMP-1 AS A MARKER FOR COLORECTAL CANCER

(76) Inventors: Wolfgang Rollinger, Polling (DE); Johann Karl, Peissenberg (DE); Norbert Wild, Geretsried/Gelting (DE); Herbert Andres, Penzberg (DE); Peter Heiss, Benediktbeuern (DE); Ursula Garczarek, Dortmund (DE); Andrea Geistanger, Muenchen (DE); Friedemann Krause, Penzberg (DE)

Correspondence Address: ROCHE DIAGNOSTICS OPERATIONS INC., 9115 Hague Road, Indianapolis, IN, 46250-0457, US

(21) Appl. No.: 12/613,760

(22) Filed: Nov. 6, 2009

Related U.S. Application Data
(63) Continuation of application No. PCT/EP2008/003642, filed on May 7, 2008.

(30) Foreign Application Priority Data
May 10, 2007 (EP) 07009396.8

(51) Int. Cl. G01N 33/573

(52) U.S. Cl. 435/7.4

(57) ABSTRACT

The present invention relates to the diagnosis of colorectal cancer. It discloses the use of protein TIMP-1 (tissue inhibitor of metalloproteinase 1) as a marker molecule in the diagnosis of colorectal cancer. It relates to a method for diagnosis of colorectal cancer from a stool sample, derived from an individual, the method involving measuring TIMP-1 in the sample. Measurement of TIMP-1 can, e.g., be used in the early detection or diagnosis of colorectal cancer.

### RELATED APPLICATIONS

This application is a continuation of PCT/EP2008/003642 filed May 7, 2008 and claims priority to EP 07009396.8 filed May 10, 2007.

### SEQUENCE LISTING

The instant application contains a Sequence Listing which has been submitted via EFS-Web and is hereby incorporated by reference in its entirety. Said ASCII copy, created on Oct. 30, 2009, is named 24232US.txt, and is 2,343 bytes in size.

### FIELD OF THE INVENTION

The present invention relates to the diagnosis of colorectal cancer. It discloses the use of the protein TIMP-1 (=tissue inhibitor of metalloproteinase 1) as a marker molecule in the diagnosis of colorectal cancer. Furthermore, it especially relates to a method for diagnosis of colorectal cancer from a stool sample, derived from an individual by measuring TIMP-1 in said sample. Measurement of TIMP-1 can, e.g., be used in the early detection or diagnosis of colorectal cancer.

### BACKGROUND

Cancer remains a major public health challenge despite progress in detection and therapy. Amongst the various types of cancer, colorectal cancer (=CRC) is one of the most frequent cancers in the Western world.

The earlier cancer can be detected/diagnosed, the better is the overall survival rate. This is especially true for CRC. The prognosis in advanced stages of tumor is poor. More than one third of the patients will die from progressive disease within five years after diagnosis, corresponding to a survival rate of about 40% for five years. Current treatment is only curing a fraction of the patients and clearly has the best effect on those patients diagnosed in an early stage of disease.

With regard to CRC as a public health problem, it is essential that more effective screening and preventative measures for colorectal cancer be developed.

The earliest detection procedures available at present for colorectal cancer involve using tests for fecal blood or endoscopic procedures. However, significant tumor size must typically exist before fecal blood is detected. With regard to detection of CRC from a stool sample, currently the most frequently used assay is the guaiac-based fecal occult blood test.

In the recent years a tremendous amount of so-called colon specific or even so-called colorectal cancer specific genes has been reported. The vast majority of the corresponding research papers or patent applications are based on data obtained by analysis of RNA expression patterns in colon (cancer) tissue versus a different tissue or an adjacent normal tissue, respectively. Such approaches may be summarized as differential mRNA display techniques.

As an example for data available from mRNA-display techniques, WO 01/96390 shall be mentioned and discussed. This application describes and claims more than two hundred isolated polynucleotides and the corresponding polypeptides as such, as well as their use in the detection of CRC. However, it is general knowledge that differences on the level of mRNA are not mirrored by the level of the corresponding proteins. A protein encoded by a rare mRNA may be found in very high amounts and a protein encoded by an abundant mRNA may nonetheless be hard to detect and find at all. This lack of correlation between mRNA-level and protein level is due to reasons like mRNA stability, efficiency of translation, stability of the protein, etc.

There also are recent approaches investigating the differences in protein patterns between different tissues or between healthy and diseased tissue in order to identify candidate marker molecules which might be used in the diagnosis of CRC. Bruenagel, G. et al., Cancer Research 62 (2002) 2437-2442, have identified seven nuclear matrix proteins which appear to be more abundant in CRC tissue as compared to adjacent normal tissue. No data from liquid and stool samples obtained from an individual are reported.

WO 02/078636 reports about nine colorectal cancer-associated spots as found by surface-enhanced laser desorption and ionization (SELDI). These spots are seen more frequently in sera obtained from patients with CRC as compared to sera obtained from healthy controls. However, the identity of the molecule(s) comprised in such spot, e.g., its (their sequence), is not known.

Despite the large and ever growing list of candidate protein markers in the field of CRC, to date clinical/diagnostic utility of these molecules is not known. In order to be of clinical utility a new diagnostic marker as a single marker should be at least as good as the best single marker known in the art. Or, a new marker should lead to a progress in diagnostic sensitivity and/or specificity either if used alone or in combination with one or more other markers, respectively. The diagnostic sensitivity and/or specificity of a test is often assessed by its receiver-operating characteristics, which will be described in detail below.

At present, for example, diagnostic blood tests based on the detection of carcinoembryonic antigen (CEA), a tumor-associated glycoprotein, are available to assist diagnosis in the field of CRC. CEA is increased in 95% of tissue samples obtained from patients with colorectal, gastric, and pancreatic cancers and in the majority of breast, lung, and head and neck carcinomas (Goldenberg, D. M. et al., J. Natl. Cancer Inst. (Bethesda) 57 (1976) 11-22). Elevated CEA levels have also been reported in patients with nonmalignant disease, and many patients with colorectal cancer have normal CEA levels in the serum, especially during the early stage of the disease (Carriquiry, L. A. and Pineyro, A., Dis. Colon Rectum 42 (1999) 921-929; Herrera, M. A. et al., Ann. Surg. 183 (1976) 5-9; Wanebo, H. J. et al., N. Engl. J. Med. 299 (1978) 448-451). The utility of CEA as measured from serum or plasma in detecting recurrences is reportedly controversial and has yet to be widely applied (Martell, R. E. et al., Int. J. Biol. Markers 13 (1998) 145-149; Moertel, C. G. et al., JAMA 270 (1993) 943-947).

In light of the available data, serum CEA determination possesses neither the sensitivity nor the specificity to enable its use as a screening test for colorectal cancer in the asymptomatic population (Reynoso, G. et al., JAMA 220 (1972) 361-365; Sturgeon, C., Clin. Chem. 48 (2002) 1151-1159).

Because metastatic disease is the main cause of cancer patient morbidity and mortality, molecules involved in the regulation of tumor invasion and metastasis are attractive as potential diagnostic/prognostic targets. It is well established that proteolytic enzymes produced by cancer cells or by cells in the tumor stroma are involved in extracellular tissue degradation, leading to cancer cell invasion and metastasis. A number of enzymes have been associated with this process, the most thoroughly investigated being the metalloproteinases, such as the collagenases and stromelysins, and the serine proteases such as plasmin.

Matrix metalloproteinases (MMP's) play a pivotal role in cancer growth and spread, contributing to enzymatic degradation of the extracellular matrix. The naturally occurring inhibitors of MMP's, are called tissue inhibitors of MMP's or TIMP's. TIMP's form tight 1:1 stoichiometric complexes with the activated forms of the MMP's thereby inhibiting the catalytic activity of these enzymes. While the balance between the matrix-degrading properties of MMP's and the inhibitory effect of TIMP's is closely regulated under normal physiological conditions (Matrisian, L. M., Bioessays 14 (1992) 455-463; Birkedal-Hansen, H., et al., Crit. Rev. Oral Biol. Med. 4 (1993) 197-250), this balance might be disrupted in malignant tissue.

A number of enzyme-linked immunoassays for the detection of TIMP-1 (Kodama, S., et al., Matrix 9 (1989) 1-6; Cooksley, S., et al., Matrix 10 (1990) 285-291; Clark, I. M., et al., Matrix 11 (1991) 76-85) and TIMP-2 (Fujimoto, N., et al., Clin. Chim. Acta 220 (1993) 31-45) have been described. These assays have been applied to body fluids, e.g. serum, plasma, amniotic fluid, cerebrospinal fluid, urine, but the number of samples tested has not been sufficient to establish normal ranges for TIMP levels in healthy individuals (Kodama, S., et al., Matrix 9 (1989) 1-6; Clark, I. M., et al., Matrix 11 (1991) 76-85). Furthermore, none of these assays has been sufficiently validated for technical performance or for clinical use.

In a study by Mimori et al. (Mimori, K., et al., Br. J. Cancer 76 (1997) 531-536) in which tumor tissue levels of TIMP-1 mRNA were studied in patients with gastric carcinoma, high tumor/normal tissue ratios of TIMP-1 mRNA were found to be associated with increased invasion and poor prognosis. However, TIMP-1 protein levels in sera from prostate cancer patients and healthy donors (Baker, T., et al., Br. J. Cancer 70 (1994) 506-512) showed a high degree of overlap. Similarly, a separate study of plasma from prostate cancer patients and healthy donors showed no difference in TIMP-1 levels, between the two groups (Jung, K., et al., Int. J. Cancer 74 (1997) 220-223).

Studies of TIMP-1 complexed with MMP-9 in plasma of patients with advanced gastrointestinal and gynaecological cancer (Zucker, S., et al., Cancer 76 (1995) 700-708) demonstrated significantly higher levels in blood samples from cancer patients with metastatic disease compared to healthy control individuals, and that patients with high levels of TIMP-1:MMP-9 complex in their circulation had a shorter survival (Zucker, S., et al., Cancer 76 (1995) 700-708, and U.S. Pat. No. 5,324,634).

Holten-Anderson M. N. (US 2003/082652) describe that TIMP-1 as measured from a bodily fluid sample can be used in the assessment of cancer. Whereas several sources of sample, including stool, are mentioned, data from blood plasma are shown. As obvious to the artisan no correlation whatsoever exists in between the concentration for a protein detected in plasma and the presence or even less so to the concentration of such protein in a stool sample. No data can be found in the art demonstrating the presence of TIMP-1 in a stool sample. No data are available in the art, indicative for the fact that the presence of TIMP-1 in a stool sample could be of clinical utility.

Samples taken from stool have the advantage that their sampling is easily possible by non-invasive means.

As mentioned above, the guaiac test is currently most widely used as a screening assay for CRC from stool. The guaiac test, however, has both poor sensitivity as well as poor specificity. The sensitivity of the guaiac-based fecal occult blood tests is ˜26%, which means that 74% of patients with malignant lesions will remain undetected (Ahlquist, D. A., Gastroenterol. Clin. North Am. 26 (1997) 41-55) in screening procedures based on the guaiac assay.

The visualization of precancerous and cancerous lesions by colonoscopy represents the best approach to early detection. Colonoscopy, however, is invasive with significant costs, risks, and complications (Silvis, S. E. et al., JAMA 235 (1976) 928-930; Geenen, J. E. et al., Am. J. Dig. Dis. 20 (1975) 231-235; Anderson, W. F. et al., J. Natl. Cancer Institute 94 (2002) 1126-1133).

The sensitivity and specificity of diagnostic alternatives to the guaiac test have been recently investigated by Sieg, A. et al., Int. J. Colorectal Dis. 14 (1999) 267-271. Especially the measurement of hemoglobin and of the hemoglobin-haptoglobin complex from stool specimen have been compared. It has been noted that the hemoglobin assay has an unsatisfactory sensitivity for the detection of colorectal neoplasms. Whereas cancer in its progressed carcinoma stage is detected with a sensitivity of about 87% the earlier tumor stages are not detected with a sufficient sensitivity. The hemoglobin-haptoglobin complex assay was more sensitive in the detection of earlier stages of CRC. This more sensitive detection was accompanied by a poor specificity. Since poor specificity, however, translates to a high number of unnecessary secondary investigations, like colonoscopy, an assay with a poor specificity also does not meet the requirements of a generally accepted screening assay.

Calprotectin has been described as an alternative biomarker for the detection of CRC from stool samples in U.S. Pat. No. 5,455,160 and correspondingly in the scientific literature by Roseth, A. G., et al. (Scand J Gastroenterol 27 (1992) 793-798; Scand. J. Gastroenterol. 28 (1993) 1073-1076). Although calprotectin is a marker of inflammatory diseases its potential as a marker for the detection of CRC from stool is documented by several publications (Johne, B., et al., Scand. J. Gastroenterol. 36 (2001) 291-296; Limburg, P. J., et al., Am. J. Gastroenterol. 98 (2003) 2299-2305; Hoff, G., et al., Gut 53 (2004) 1329-1333). While the sensitivity and specificity of calprotectin are comparable to the immunological hemoglobin assay, calprotectin appears to have some characteristics favorable for a diagnostic biomarker as compared to hemoglobin. It is evenly distributed in feces, it is stable at room temperature making mail delivery of the sample to the laboratory feasible and it shows no interference with food components or pharmaceutical compounds (Ton, H., et al., Clin. Chim. Acta 292 (2000) 41-54). However, elevated concentrations of calprotectin, the heterodimer of S100A8 and S100A9, were detected in stool samples from patients suffering from CRC, Crohn's disease or inflammatory bowel disease. These results are in agreement with the more general role of calprotectin in inflammation (Ryckman, C., et al., J. Immunol. 170 (2003) 3233-3242). Hence, the use of calprotectin in gastroenterology is not limited to the detection of CRC but extends to other diseases, especially inflammatory bowel disease as reviewed by Poullis, A., et al. (J. Gastroenterol. Hepatol. 18 (2003) 756-762).

Recently, an assay for detection of pyruvate kinase M2 isoenzyme (M2-PK) has been introduced into the market (Schebo Biotech, Gieβen, Germany). A comparison of the guaiac assay to the immuno assays for hemoglobin and M2-PK has for example been performed by Vogel, T. et. al., Dtsch. Med. Wochenschr. 130 (2005) 872-877. They show that the immunological assays are superior to the guaiac test and that at comparable specificity the M2-PK assay is less sensitive in detecting CRC as compared to the hemoglobin assay. Yet, the authors conclude that the usefulness of both these stool based assays is still questionable.

A further alternative method to the guaiac test for detection of CRC in stool has been published recently and consists in the detection of the colorectal cancer-specific antigen, “minichromosome maintenance protein 2” (MCM2) by immunohistochemistry in colonic cells shed into stool. Due to the small study size, conclusion on the diagnostic value for detection of colorectal cancer is preliminary. However, the test seems to have only limited sensitivity to detect right-sided colon cancer (Davies, R. J. et al., Lancet 359 (2002) 1917-1919).

Osborn, N. K. and Ahlquist, D. A., (Gastroenterology 128 (1995) 192-206) discuss the disadvantages of the marker hemoglobin and strongly favor the use of DNA recovered from a stool sample in the screening for CRC.

The identification of an early CRC tumor marker that would allow reliable cancer detection or provide early prognostic information by non-invasive means from a stool specimen could lead to a diagnostic assay that would greatly aid in the diagnosis and in the management of this disease. Therefore, an urgent clinical need exists to improve the diagnosis of CRC, especially from stool. It is especially important to improve the early diagnosis of CRC, since for patients diagnosed early on chances of survival are much higher as compared to those diagnosed at a progressed stage of disease.

It was the task of the present invention to investigate whether a new marker can be identified which may aid in CRC diagnosis. Preferably such marker would be present in stool and allow for a non-invasive diagnosis.

Surprisingly, it has been found that the use of the protein TIMP-1 as a marker polypeptide for CRC can at least partially overcome the problems known from the state of the art.

### SUMMARY OF THE INVENTION

The present invention therefore relates to a method for the diagnosis of colorectal cancer comprising the steps of
(a) providing a stool sample obtained from an individual,
(b) contacting said sample with a specific binding agent for TIMP-1 under conditions appropriate for formation of a complex between said binding agent and TIMP-1,
(c) determining the amount of complex formed in step (b), and
(d) correlating the amount of complex determined in (c) to the diagnosis of colorectal cancer.

As a further preferred embodiment the present invention discloses a method for diagnosing colorectal cancer comprising the steps of providing a stool sample obtained from an individual, contacting said sample with a specific binding agent for TIMP-1 under conditions appropriate for formation of a complex between said binding agent and TIMP-1, contacting said sample with a specific binding agent for at least one second marker selected from the group consisting of hemoglobin/haptoglobin complex, hemoglobin, calprotectin, and tumor M2 pyruvate kinase (M2-PK) under conditions appropriate for formation of a complex between said binding agent and the second marker, detection the amount of complex formed for TIMP-1 and the at least one second marker and correlating the amount of complexes formed in steps to the diagnosis of colorectal cancer. In further preferred embodiments the method according to the present invention is based on the determination and of TIMP-1 and of hemoglobin as the second marker, the determination of TIMP-1 and of the hemoglobin/haptoglobin complex as the second marker, the determination of TIMP-1 and of calprotectin as the second marker, and the determination of TIMP-1 and of M2-PK as the second marker, respectively.

As obvious to the skilled artisan the measurement of TIMP-1 and the measurement of optionally one or more other marker is made from an aliquot of a stool sample. Such measurement or measurements can be made from the same aliquot of a stool sample or of a processed stool sample, respectively, or from different aliquots of a patient's stool sample or from different aliquots of a patient's processed stool sample, respectively.

As the skilled artisan will appreciate, any such measurement of TIMP-1 from a stool sample is made in vitro. The patient sample is discarded afterwards. The patient sample is solely used for the in vitro method of the invention. Neither the measurement of TIMP-1 nor the assessment of CRC is performed on the human or animal body.

The in vitro diagnostic procedure according to the present invention is used to assess the absence, the presence or the relative concentration of TIMP-1 in a stool sample. The value measured for TIMP-1 will aid the clinician in assessing CRC, e.g., in his establishing a clinical diagnosis and/or in his decision for an appropriate treatment. If a relative concentration of TIMP-1 is used in the assessment of CRC such relative concentration of TIMP-1 is most easily and preferably based on the ratio of the amount of TIMP-1 per amount of stool.

In a preferred embodiment the stool sample is processed to obtain a processed sample liquid which is more convenient to handle than a stool specimen. Such processed sample is then incubated with the specific binding agent for TIMP-1. The present invention therefore also relates to a method for the diagnosis of colorectal cancer comprising the steps of
(a) providing a stool sample obtained from an individual,
(b) processing said sample to obtain a processed liquid sample,
(c) contacting said processed liquid sample with a specific binding agent for TIMP-1 under conditions appropriate for formation of a complex between said binding agent and TIMP-1, and
(d) correlating the amount of complex formed in (c) to the diagnosis of colorectal cancer.

Another preferred embodiment of the invention is a method for the diagnosis of colorectal cancer comprising the steps of
(a) processing a stool sample obtained from an individual to obtain a processed liquid sample,
(b) contacting said processed liquid sample with a specific binding agent for TIMP-1 under conditions appropriate for formation of a complex between said binding agent and TIMP-1,
(c) determining the amount of complex formed in step (b), and
(d) correlating the amount of complex determined in (c) to the diagnosis of colorectal cancer.

### DESCRIPTION OF THE FIGURE

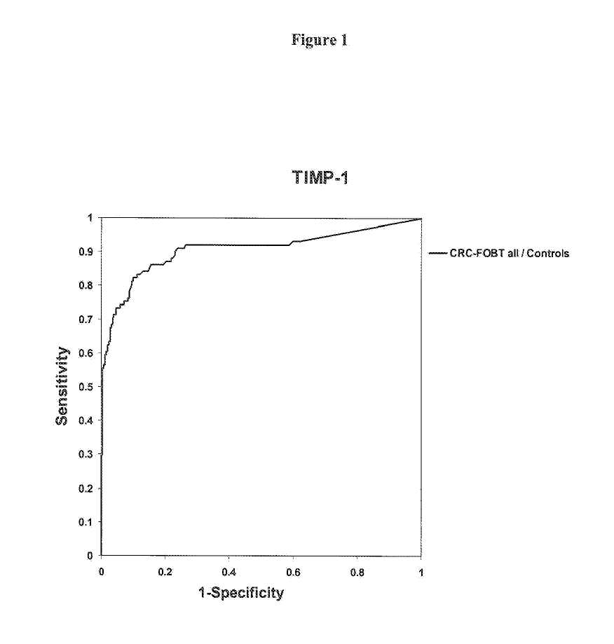

FIG. 1: ROC for TIMP-1
The receiver operator curve (ROC) for the assessment of 101 samples obtained from patients with CRC as compared to 252 samples obtained from screening of healthy (=control) individuals is given.

### DETAILED DESCRIPTION OF THE INVENTION

The protein TIMP-1 (=tissue inhibitor of metalloprotease 1) is characterized by the sequence given SEQ ID NO: 1.

In a preferred embodiment, the novel marker TIMP-1 is used in the screening of individuals for CRC.

In a preferred embodiment the diagnostic method according to the present invention is used for screening purposes, i.e., it is used to assess subjects without a prior diagnosis of CRC by measuring the level of TIMP-1 in a stool sample and correlating the level measured to the presence or absence of CRC.

As the skilled artisan will readily appreciate, in such screening care has to be taken that an appropriate amount of a stool sample is used. Preferably a defined amount of stool sample is used for the measurement of TIMP-1 comprised therein. Preferably the amount of TIMP-1 is expressed in terms of amount of TIMP-1 per amount of stool, i.e., the relative concentration of TIMP-1 is given.

As the skilled artisan will as well appreciate a cut off-value for TIMP-1 is established based on TIMP-1 values as measured in the stool sample derived from individuals of a healthy normal population. The clinically relevant normal population in a screening setting preferably consists of clinically healthy individuals in the age of 55 to 65 years. A TIMP-1 value in a stool sample above an established cut-off value may be considered indicative for CRC or may at least warrant further diagnostic examination of the respective individual.

Colorectal cancer most frequently progresses from adenomas (polyps) to malignant carcinomas.

The staging of cancer is the classification of the disease in terms of extent, progression, and severity. It groups cancer patients so that generalizations can be made about prognosis and the choice of therapy.

The different stages of CRC used to be classified according to Dukes' stages A to D. Today, the TNM system is the most widely used classification of the anatomical extent of cancer. It represents an internationally accepted, uniform staging system. There are three basic variables: T (the extent of the primary tumor), N (the status of regional lymph nodes) and M (the presence or absence of distant metastases). The TNM criteria are published by the UICC (International Union Against Cancer), Sobin, L. H., Wittekind, Ch. (eds.), TNM Classification of Malignant Tumours, sixth edition, 2002). Once the TNM status is determined the patients are grouped into disease stages that are denoted. by Roman numerals ranging form I to IV with IV being the most advanced disease stage. TNM staging and UICC disease stages correspond to each other as shown in the following table taken from Sobin L. H. and Wittekind (eds.) supra.

### Interrelation of TNM Staging and UICC Disease Stages

| UICC disease stage | T staging | N staging | M staging |
|---|---|---|---|
| Stage 0 | Tis | N0 | M0 |
| Stage I | T1, T2 | N0 | M0 |
| Stage IIA | T3 | N0 | M0 |
| Stage IIB | T4 | N0 | M0 |
| Stage IIIA | T1, T2 | N1 | M0 |
| Stage IIIB | T3, T4 | N1 | M0 |
| Stage IIIC | Any T | N2 | M0 |
| Stage IV | Any T | Any N | M1 |

What is especially important is, that early diagnosis of CRC translates to a much better prognosis. Malignant tumors of the colorectum arise from benign tumors, i.e. from adenoma. Therefore, best prognosis have those patients diagnosed at the adenoma stage. Patients diagnosed as early as in stage N0, M0 or T1-3; N0; M0, if treated properly have a more than 90% chance of survival 5 years after diagnosis as compared to a 5-years survival rate of only 10% for patients diagnosed when distant metastases are already present.

In the sense of the present invention early diagnosis of CRC refers to a diagnosis at a tumor stage where no metastases at all (neither proximal=N0, nor distal=M0) are present, i.e. the stages of, Tᵢₛ, N0, M0 or T1-4; N0; M0. Tᵢₛ denotes carcinoma in situ.

It is preferred, that CRC is diagnosed when it has not yet fully grown through the bowel wall and thus neither the visceral peritoneum is perforated nor other organs or structures are invaded, i.e., that diagnosis is made at any stage from T₁ₛ; N0; M0 to T3; N0; M0 (=Tᵢₛ-3; N0; M0).

The diagnostic method according to the present invention is based on a stool sample which is derived from an individual. The stool sample is extracted and TIMP-1 is specifically measured from this processed stool sample by use of a specific binding agent.

A specific binding agent is, e.g., a receptor for TIMP-1, a lectin binding to TIMP-1, an aptamer to TIMP-1, or an antibody to TIMP-1. A specific binding agent has at least an affinity of 10⁷ l/mol for its corresponding target molecule. The specific binding agent preferably has an affinity of 10⁸ l/mol or even more preferred of 10⁹ l/mol for its target molecule. As the skilled artisan will appreciate the term specific is used to indicate that other biomolecules present in the sample do not significantly bind to with the binding agent specific for TIMP-1. Preferably, the level of binding to a biomolecule other than the target molecule results in a binding affinity which is only 10%, more preferably only 5% of the affinity of the target molecule or less. A most preferred specific binding agent will fulfill both the above minimum criteria for affinity as well as for specificity.

A specific binding agent preferably is an antibody reactive with TIMP-1. The term antibody refers to a polyclonal antibody, a monoclonal antibody, fragments of such antibodies, as well as genetic constructs comprising the binding domain of an antibody. Any antibody fragment retaining the above criteria of a specific binding agent can be used. Antibodies are generated by state of the art procedures, e.g., as described in Tijssen (Tijssen, P., Practice and theory of enzyme immunoassays, Elsevier, Amsterdam (1990), the whole book, especially pp. 43-78). In addition, the skilled artisan is well aware of methods based on immunosorbents that can be used for the specific isolation of antibodies. By these means the quality of polyclonal antibodies and hence their performance in immunoassays can be enhanced (Tijssen, P., supra, pages 108-115).

For the achievements as disclosed in the present invention polyclonal antibodies raised in rabbits can be used. However, clearly also polyclonal antibodies from different species , e.g. rats or guinea pigs, as well as monoclonal antibodies can also be used. Since monoclonal antibodies can be produced in any amount required with constant properties, they represent ideal tools in development of an assay for clinical routine. The generation and use of monoclonal antibodies to TIMP-1 in a method according to the present invention is yet another preferred embodiment.

As the skilled artisan will appreciate now, that TIMP-1 has been identified by an immuno assay procedure as a marker which is useful in the diagnosis of CRC, alternative ways may be used to reach a result comparable to the achievements of the present invention. The marker protein TIMP-1 may be detected by any appropriate means and used as a marker of CRC. Such preferred appropriate means comprise the detection of the TIMP-1 polypeptide by an immuno assay procedure, by liquid chromatography, especially high performance liquid chromatography, by electrophoresis, especially SDS-PAGE combined with Western Blotting and by mass spectroscopy.

For measurement, the stool sample is obtained from an individual. An aliquot of the stool sample may be used directly. Preferably an aliquot of the stool sample is processed to yield a liquid sample. A processed stool sample is a liquid sample obtained upon extraction of a stool sample with an extraction buffer.

Any appropriate extraction buffer may be used. An appropriate extraction buffer should fulfill at least three basic requirements: It should liberate the analyte of interest from the stool matrix. It should stabilize the free analyte. It should minimize the interference of the stool matrix in the subsequent detection of the analyte. In one embodiment the processing of the stool sample is accomplished by an extraction buffer that is optimized for the task. Since marker combinations might hold additional diagnostic potential, an optimized buffer should not only be applicable for one specific biomarker but for all analytes of interest. The extraction buffer may contain urea to improve the homogenization and extraction of the stool sample. Ca²⁺ may be included for stabilization of a Ca²⁺-binding protein. Weak chelating agents should be used that on the one hand can break ion bridges between Ca²⁺-binding proteins and the stool matrix. An optimized and preferred extraction buffer contains urea, Ca²⁺-ions and a chelator. Preferably the chelator is selected from the group consisting of nitrilotriacetic acid or citrate.

It is most convenient to use an optimized extraction buffer in combination with a tailor-made stool sampling device. In a most convenient way, an individual collects a defined amount of stool sample and transfers it directly into the collection prefilled with the stabilizing extraction buffer. This convenient mode of sampling and extraction enables the transport of the specimen to a diagnostic laboratory without degradation of the analyte. Since the extraction of the stool sample can be achieved directly in the sampling device the necessary handling and transfer procedures are reduced.

Several recent developments have focused on devices that facilitate the sampling and handling of a stool sample. EP 1 366 715 discloses a special collection tube for collection of a stool sample. This extraction tube essentially comprises (a) a container body that is hollow on the inside, open at the top, and able to receive a buffer solution, (b) a top cap provided with a threaded small rod for collection of fecal samples, said threaded small rod protruding axially inside the container body, when the top cap is applied to the top end of the container body, and (c) a dividing partition provided, in an intermediate position, inside said container body so as to separate a top chamber from a bottom chamber inside said container body, said dividing partition having an axial hole suitable to allow the passage of said threaded small rod, so as to retain the excess feces in said top chamber and allow the passage of the threaded part of the small rod into said bottom chamber. This extraction tube further has a container body that is open at the bottom and provided with a bottom cap which can be applied movably to the bottom end of the container body, so that said extraction tube can be used directly as a primary sampling tube to be inserted into a sample-holder plate of automatic analyzers, following removal of said bottom cap and overturning of said container body. The device disclosed in EP 1 366 715 allows for the convenient handling of a defined quantity of a stool sample and has the advantage that after appropriate extraction the tube may be directly placed into the sample-holder of an automatic analyzer.

A second example of a sophisticated stool sampling device that is appropriate for a convenient sampling and handling of a stool sample is described in WO 03/068398.

The stool sample is preferably used or processed directly after sampling or stored cooled or more conveniently stored frozen. Frozen stool samples can be processed by thawing, followed by dilution in an appropriate buffer, mixing and centrifugation. Supernatants are used as liquid sample for subsequent measurement of the marker TIMP-1.

An aliquot of the processed stool sample is incubated with the specific binding agent for TIMP-1 under conditions appropriate for formation of a binding agent TIMP-1-complex. Such conditions need not be specified, since the skilled artisan without any inventive effort can easily identify such appropriate incubation conditions.

As a final step according to the method disclosed in the present invention the amount of complex is measured and correlated to the diagnosis of CRC. As the skilled artisan will appreciate there are numerous methods to measure the amount of the specific binding agent TIMP-1-complex all described in detail in relevant textbooks (cf., e.g., Tijssen P., supra, or Diamandis, E. P., and Christopoulos, T. K. (eds.), Immunoassay, Academic Press, Boston (1996)).

Preferably TIMP-1 is detected in a sandwich type assay format. In such assay a first specific binding agent is used to capture TIMP-1 on the one side and a second specific binding agent, which is labeled to be directly or indirectly detectable is used on the other side. Preferably an assay set-up is chosen that ensures that the sum of free and complexed TIMP-1 is measured.

In the assessment of CRC antibodies to TIMP-1 can also be used in other procedures, e.g., to detect colorectal cancer cells in situ, in biopsies, or in immunohistological staining procedures.

Preferably, an antibody to TIMP-1 is used in a qualitative (TIMP-1 present or absent) or quantitative (TIMP-1 amount is determined) immunoassay.

As mentioned above, it has surprisingly been found that TIMP-1 can be measured from a stool sample obtained from an individual sample. No tissue and no biopsy sample is required to apply the marker TIMP-1 in the diagnosis of CRC.

Whereas application of routine proteomics methods to tissue samples, e.g. by comparing healthy and cancerous tissue, leads to the identification of many potential marker candidates for the tissue/disease selected, these marker candidates only accidentally and in rare cases are found in the circulation. Surprisingly, the inventors of the present invention have been able to detect the protein TIMP-1 in a stool sample. They have been able to demonstrate that the presence of TIMP-1 in such stool sample obtained from an individual can be correlated to the diagnosis of colorectal cancer.

It has also been found that the presence or absence of TIMP-1 in such stool sample obtained from an individual can be correlated to the presence or absence of colorectal cancer in a patient. The present invention also relates to a method for excluding colorectal cancer comprising the steps of a) providing a stool sample obtained from an individual, b) processing said sample to obtain a processed liquid sample, c) contacting said processed liquid sample with a specific binding agent for TIMP-1 under conditions appropriate for formation of a complex between said binding agent and TIMP-1, and d) using the absence of a complex in (c) as an indicator for the absence of colorectal cancer.

As the skilled artisan will appreciate, a positive result for TIMP-1 in a stool sample must not necessarily mean that the patient has CRC. However, a positive value for TIMP-1 in a stool sample should be considered a clear-cut indicator to warrant further and more sophisticated diagnostics. In a preferred embodiment a positive value for TIMP-1 as measured from a stool sample is used as an indicator that the patient should be offered further investigations, especially a PET scan or a colonoscopy. Preferably, a positive value in a stool sample is used as an indicator that colonoscopy as the next step in the (diagnostic) examination of a patient is warranted.

In a preferred embodiment, the novel marker TIMP-1 is used for monitoring of CRC patients.

When used in patient monitoring the diagnostic method according to the present invention may help to assess tumor load, efficacy of treatment and tumor recurrence in the follow-up of patients. Increased levels of TIMP-1 are directly correlated to tumor burden. After chemotherapy a short term (few hours to 14 days) increase in TIMP-1 may serve as an indicator of tumor cell death. In the long term follow-up of patients after surgery and/or chemotherapy (from 3 months to 10 years) an increase of TIMP-1 can be used as an indicator for tumor recurrence in the colorectum.

Measuring the level of protein TIMP-1 has proven very advantageous in the field of CRC. Therefore, in a further preferred embodiment, the present invention relates to use of protein TIMP-1 as a marker molecule in the diagnosis of colorectal cancer from a stool sample obtained from an individual.

The term marker molecule is used to indicate that an increased level of the analyte TIMP-1 as measured from a processed stool sample obtained from an individual marks the presence of CRC. The marker molecule TIMP-1 is present in stool in free form and in the form of a TIMP-1/MMP complex. In a diagnostic method according to the present invention free TIMP-1, the TIMP-1/MMP complex or total TIMP-1 (the sum of free TIMP-1 and TIMP-1 in a TIMP-1/MMP complex) can be used as a marker molecule. Preferably total TIMP-1 is determined and the amount measured used to assess CRC.

As obvious to the skilled artisan, the present invention shall not be construed to be limited to the measurement of the full-length protein TIMP-1 of SEQ ID NO: 1. Physiological fragments of TIMP-1 can also be measured and used as a marker for CRC while practicing the present invention. Immunologically detectable fragments preferably comprise at least 6, 7, 8, 10, 12, 15 or 20 contiguous amino acids of said marker polypeptide. One of skill in the art would recognize that proteins which are released by cells or present in the extracellular matrix may be damaged, e.g., during inflammation, and could become degraded or cleaved into such fragments. In addition, or in the alternative the TIMP-1 polypeptide may carry a post-translational modification, and such modified TIMP-1 may also serve as a marker of CRC.

An assay for TIMP-1 can be set up to measure the total amount of TIMP-1, i.e. TIMP-1 bound to a matrix metalloprotease plus the amount of free TIMP-1. In this preferred embodiment at least one specific binding agent is used that binds to both free TIMP-1 as well as to TIMP-1 bound to an MMP, respectively. For example, an antibody can be used that binds to free TIMP-1 and as well to TIMP-1 in a TIMP-1/MMP complex. In case a sandwich assay format is used, a second antibody meeting the same requirements is used. In a preferred embodiment the methods according to the present invention are practiced by measuring total TIMP-1 in a stool sample.

An assay for TIMP-1 can also be set up to only measure free TIMP-1, i.e. TIMP-1 not bound to a matrix metalloprotease. In a preferred embodiment free TIMP-1 is used as a marker for CRC. In assay for measurement of free TIMP-1 at least one specific binding agent is used that only binds to free TIMP-1. For example an antibody can be used that binds to free TIMP-1 but not to TIMP-1 in a TIMP-1/MMP complex.

As illustrated above TIMP-1 forms 1:1 complexes with matrix metalloproteases. In a preferred embodiment according to the present invention a TIMP-1/MMP complex is used as a marker of CRC. In such an assay a specific binding agent to TIMP-1 can be used as a capture reagent and a specific binding agent to MMP can be used as a detection agent, or vice versa. Alternatively total and free TIMP-1 could be determined and the amount of TIMP-1 in a TIMP-1/MMP complex calculated as the difference of these two measurements.

Artificial fragments of TIMP-1 may be used, e.g. as a positive control in an immuno assay or as an immunogen. Artificial fragments preferably encompass a peptide produced synthetically or by recombinant techniques consisting of at least 6, 7, 8, 9, 10, 12, or at least 15 contiguous amino acids as derived from the sequence disclosed in SEQ ID NO: 1. Preferably such artificial fragment comprises at least one epitope of diagnostic interest. Also preferred the artificial fragment comprises at least two epitopes of interest and is appropriate for use as a positive control in a sandwich immunoassay.

It is preferred to use the novel marker TIMP-1 in the early detection of colorectal cancer. However, as the skilled artisan will appreciate TIMP-1, alike other markers, will also be of great advantage in the diagnosis and follow-up of patients already suffering from CRC at more advanced stages of tumor progression.

The TIMP-1 concentration closely correlates with tumor burden in CRC. The marker is therefore also suitable for the follow-up of CRC patients after treatment. In a preferred embodiment, the novel marker TIMP-1 is used in the follow-up of patients suffering from CRC. By measuring CRC regularly, e.g., at 3-monthly, 6-monthly, or yearly intervals, tumor progression and/or as the case may be tumor recurrence can be assessed. An increase or a re-appearance of TIMP-1 above the normal cut-off value are considered indicative for tumor progression or for tumor recurrence, respectively. A further preferred embodiment therefore relates to a method of assessing by an in vitro measurement a patient suffering from colorectal cancer after surgery for removal of the cancerous lesion the method comprising the steps of a) providing a stool sample obtained from said patient, b) contacting said sample with a specific binding agent for TIMP-1 under conditions appropriate for formation of a complex between said binding agent and TIMP-1, c) determining the amount of complex formed in step b), and d) correlating the amount of complex determined in c) to a recurrence or progression of colorectal cancer. An increase of TIMP-1 after therapy is indicative of the recurrence of CRC in the respective patient. Measurement of TIMP-1 from a stool sample is especially helpful and in a preferred embodiment used in the early detection of a CRC tumor recurrence within the gastrointestinal tract.

The colon as well as the rectum both are part of the gastrointestinal tract. Now that it has been shown that TIMP-1 most likely will be useful in the screening of patients for colorectal cancer, it is very likely that the presence of TIMP-1 in a stool sample may also be used as a diagnostic aid in the assessment of other types of gastrointestinal cancer. In a further preferred embodiment the present invention relates to the use of TIMP-1 as measured from a stool sample in the assessment of a gastrointestinal tumor. Preferably TIMP-1 as measured from a stool sample is also used in the assessment a stomach tumor.

The use of protein TIMP-1 itself, exhibits a surprisingly good performance both in terms of sensitivity as well as specificity. Combining measurements of TIMP-1 with other known markers, like hemoglobin or the hemoglobin-haptoglobin complex, or with other markers of CRC yet to be discovered, leads and may lead, respectively, to further improvements in the assessment of CRC. Therefore in a further preferred embodiment the present invention relates to the use of TIMP-1 as a marker molecule for colorectal cancer in combination with one or more other marker molecules for colorectal cancer in the diagnosis of colorectal cancer from a stool sample obtained from an individual. Preferred selected other CRC markers with which the measurement of TIMP-1 may be combined are calprotectin, tumor M2 pyruvate kinase (M2-PK), hemoglobin and/or the hemoglobin-haptoglobin complex.

As a further preferred embodiment the present invention discloses the use of protein TIMP-1 as a marker molecule for colorectal cancer in combination with one or more other marker molecule(s) for colorectal cancer selected from the group consisting of hemoglobin/haptoglobin complex, hemoglobin, calprotectin, and tumor M2 pyruvate kinase (M2-PK).

In a preferred embodiment the present invention relates to the use of a marker combination comprising the markers TIMP-1 and hemoglobin in the assessment of CRC, whereas both markers are measured from a stool sample.

In a preferred embodiment the present invention relates to the use of a marker combination comprising the markers TIMP-1 and the hemoglobin/haptoglobin complex in the assessment of CRC, whereas both markers are measured from a stool sample.

Diagnostic reagents in the field of specific binding assays, like immunoassays, usually are best provided in the form of a kit, which comprises the specific binding agent and the auxiliary reagents required to perform the assay. The present invention therefore also relates to an immunological kit comprising at least one specific binding agent for TIMP-1 and auxiliary reagents for measurement of TIMP-1.

Accuracy of a diagnostic test is often described by its receiver-operating characteristics (ROC) (see especially Zweig, M. H. and Campbell, G., Clin. Chem. 39 (1993) 561-577). The ROC graph is a plot of all of the sensitivity/specificity pairs resulting from continuously varying the decision thresh-hold over the entire range of data observed.

The clinical performance of a laboratory test depends on its diagnostic accuracy, or the ability to correctly classify subjects into clinically relevant subgroups. Diagnostic accuracy measures the test's ability to correctly distinguish two different conditions of the subjects investigated. Such conditions are for example health and disease or benign versus malignant disease.

In each case, the ROC plot depicts the overlap between the two distributions by plotting the sensitivity versus 1−specificity for the complete range of decision thresholds. On the y-axis is sensitivity, or the true-positive fraction [defined as (number of true-positive test results)/(number of true-positive+number of false-negative test results)]. This has also been referred to as positivity in the presence of a disease or condition. It is calculated solely from the affected subgroup. On the x-axis is the false-positive fraction, or 1−specificity [defined as (number of false-positive results)/(number of true-negative+number of false-positive results)]. It is an index of specificity and is calculated entirely from the unaffected subgroup. Because the true- and false-positive fractions are calculated entirely separately, by using the test results from two different subgroups, the ROC plot is independent of the prevalence of disease in the sample. Each point on the ROC plot represents a sensitivity/1-specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. (If the ROC plot falls completely below the 45° diagonal, this is easily remedied by reversing the criterion for “positivity” from “greater than” to “less than” or vice versa.) Qualitatively, the closer the plot is to the upper left corner, the higher the overall accuracy of the test.

One convenient goal to quantify the diagnostic accuracy of a laboratory test is to express its performance by a single number. The most common global measure is the area under the curve of the ROC plot. By convention, this area is always ≧0.5 (if it is not, one can reverse the decision rule to make it so). Values range between 1.0 (perfect separation of the test values of the two groups) and 0.5 (no apparent distributional difference between the two groups of test values). The area does not depend only on a particular portion of the plot such as the point closest to the diagonal or the sensitivity at 90% specificity, but on the entire plot. This is a quantitative, descriptive expression of how close the AUC is to the perfect one (area=1.0).

Clinical utility of the novel marker TIMP-1 has been assessed in comparison to and in combination with the established marker hemoglobin using a receiver operator characteristics analysis (ROC; Zweig, M. H. and Campbell, G., Clin. Chem. 39 (1993) 561-577). This analysis has been based on samples derived from well-defined patient cohorts as given in the examples section.

Surprisingly it could be demonstrated that a marker combination based on the values of TIMP-1 and of hemoglobin—both measured from a stool sample—exhibits an improved diagnostic accuracy as compared to each of them if used as a single marker, respectively.

The following examples, figure and sequence listing are provided to aid the understanding of the present invention, the true scope of which is set forth in the appended claims. It is understood that modifications can be made in the procedures set forth without departing from the spirit of the invention.

### Example 1

### Study Population

In order to obtain a high number of clinically well-characterized stool samples a prospective multi-center study was initiated. The participants received detailed instructions on how the fecal samples had to be collected. Each patient had to collect two different portions of one singular feces sample at different sites and to freeze both within one day at −20° C. The stool sample had to be collected before the colonoscopy took place (control collective) or before the surgery (cancer collective) was performed. After storage at −20° C. for a maximum of 2 weeks the stool samples were stored at −70° C. until the stool extraction was carried out. The patients for the control collective were recruited at gastroenterology units in an average-risk screening population, which underwent a colonoscopy. Patients with inflammatory bowel diseases and with any kind of adenomas were excluded from the control collective. Due to the low prevalence of colorectal cancer patients within the preventive screening population the cancer patients were collected at different surgery units. The diagnosis of colorectal cancer was confirmed by the pathological staging of each cancer patient. In order to avoid a positive bias for the immunochemical FOBT all cancer patients were excluded, from which a guaiac-based FOBT was performed before the colonoscopy or which were detected due to visible rectal bleeding. In total 252 control patients and 101 colorectal cancer patients were included into the study (Table 1).

TABLE 1
Baseline characteristics of the sample collectives

| | Total | | Gender |
|---|---|---|---|
| | number | Age (year) | (female/male) |
| Controls | 252 | 63.0 +/− 8.0 | 151/101 |
| Healthy Controls | 132 | 62.3 +/− 6.8 | 81/51 |
| (no evidence of any | | | |
| bowel disease) | | | |
| Hemorrhoids | 28 | 60.1 +/− 7.1 | 13/15 |
| Diverticulosis | 73 | 64.7 +/− 9.5 | 46/27 |
| Hyplastic polyps | 14 | 67.9 +/− 9.9 | 8/6 |
| Other GI diseases | 5 | 59.2 +/− 6.7 | 3/2 |
| CRC (all stages) | 101 | 68.4 +/− 11.5 | 48/53 |
| UICC 0 | 1 | — | 0/1 |
| UICC I | 22 | 65.9 +/− 9.3 | 8/14 |
| UICC II | 27 | 73.1 +/− 10.9 | 14/13 |
| UICC III | 12 | 70.9 +/− 12.3 | 8/4 |
| UICC IV | 23 | 69.6 +/− 10.3 | 12/11 |
| w/o staging | 16 | 61.9 +/− 12.6 | 6/10 |

### Example 2

### Extraction of the Stool Samples for the Determination of Hemoglobin and TIMP-1

From each patient two aliquots are collected at different sites of a single stool samples. About 1 g of stool per sample is collected by the patients in a special sampling device (Sarstedt, Germany, order-no.: 80 623 022), frozen within one day and stored at −70° C. until extraction.

For the processing of the stool samples an extraction buffer is freshly prepared by adding a protease inhibitor cocktail (Mini Complete EDTA-free, Roche, Germany, order-no.: 11 873 580) to the following buffer:

| TRIS | 0.10 mol/l, pH 8.0 |
|---|---|
| Citric acid | 0.10 mol/l |
| Urea | 1.00 mol/l |
| CaCl₂ | 0.01 mol/l |
| BSA | 0.50% |

The stool samples are thawed and 50-100 mg of each sample are transferred to a fecal sample preparation kit (Roche, Germany, order-no.: 10 745 804) using the disposable spatula of the device. Extraction buffer is added according to the weight of the stool sample to give a 50-fold dilution. The samples are vigorously mixed on an orbital shaker for 30 minutes, transferred to a 10 ml tube (Sarstedt, Germany, order-no.: 62 551 201) and centrifuged at 1,200 g for 10 minutes. The supernatant is filtered using a 5 μm cut-off filter (ULTRAFREE-CL, Millipore, Germany, order-no. UFC40SV25), aliquoted and stored for further analysis at −70° C. These extracts are used for measuring all biomarkers of interest in this study.

### Example 3

### Analyte Stability of TIMP-1 in Stool Extract

The determination of TIMP-1 is performed with the “QUANTIKINE human TIMP-1 Immunoassay (Cat. No DTM100; R&D Systems, Minneapolis) basically according to the instructions given by the manufacturer for measurement of TIMP-1 in a serum or plasma sample. However, in order to be able to measure TIMP-1 in stool, stool extracts (see Example 2) are diluted in a ratio of 1:2 with the Calibrator diluent RD5P of the kit. These pre-diluted stool extracts are then used as a sample as described in the package insert (50 μl of the pre-diluted stool extract+100 μL, of Assay Diluent RD1X). This commercial TIMP-1 assay employs a monoclonal anti-TIMP-1 antibody as a capture reagent and a polyclonal antibodies against TIMP-1 as a detection reagent and thus detects total TIMP-1 in a sample.

The hemoglobin determination is performed with the “RIDASCREEN Hemoglobin” assay (Cat. No G09030, R-Biopharm AG, Darmstadt) according to the instructions given by the manufacturer. 10 μL of the above described stool extract is used and diluted with 90 μL sample diluent of the assay (1:10 dilution ratio).

20 Stool samples with analyte concentrations in the critical range at or above the cut-off value are freshly extracted as described above and then stored for 1 or 3 days at room temperature. Of the 20 samples used to assess the stability 18 are positive for hemoglobin and 7 are positive for TIMP-1 and can be used for calculation of the analyte stability.

TABLE 2
Recovery of TIMP-1 and hemoglobin in stool extracts after temperature stress

| | | Concentration | Recovery after 1 d at | Recovery after 3 d at |
|---|---|---|---|---|
| | N | range of samples | RT (median +/− SD) | RT (median +/− SD) |
| TIMP-1 | 7 | 1.0 ng/g-17.6 ng/g | 96.7% | 99.7% |
| Hemoglobin | 18 | 0.32 μg/g-10.4 μg/g | 79.1% | 58.6% |

As is obvious from Table 2, TIMP-1 appears to be extremely stable in stool extracts prepared as described above and is significantly more stable than hemoglobin. This is a great advantage, especially if the stool extraction buffer is incorporated into a tailor-made stool sample device. This way a stool sample can be collected directly into a device containing the extraction buffer, the stool extract is prepared immediately within the device, and the tube containing the sample to be analyzed can be sent to the lab at room temperature.

### Example 4

### Clinical Utility of TIMP-1 in Diagnosis of Colorectal Cancer

The clinical utility of TIMP-1 is assessed by analyzing stool samples obtained from a well-characterized patient cohort described in Example 1. For each patient two stool samples from the same bowel movement are measured and the concentrations are analyzed. The correlation of both concentrations is assessed by Pearson's correlation coefficient. This analysis reveals a close correlation between the two extracts. To improve the sensitivity of the assay the maximum concentration measured in one of the two paired samples is used for further analysis. The diagnostic value of TIMP-1 is evaluated by ROC analysis according to Zweig et al (supra). Discriminatory power for differentiating patients in the CRC group from healthy individuals as measured by the area under the curve is found to be 91% for CRC vs. the screening population (FIG. 1).

To calculate the sensitivity for the marker TIMP-1 in the early detection of CRC, the cut-off for positivity was set to result in either 95% or 98% specificity, respectively, as compared to the control collective.

TABLE 3
ROC analysis, sensitivity and specificity of TIMP-1

| Sample panel | N | ROC area % |
|---|---|---|
| CRC samples w/o blood/FOBT | 101 | 91 |
| UICC stage I | 23 | 85 |
| UICC stage II | 27 | 95 |
| UICC stage III | 12 | 96 |
| UICC stage IV | 23 | 90 |
| Cut-off: | | |
| 95% specificity | | 23.8 ng/g stool |
| 98% specificity | | 44.7 ng/g stool |
| Sensitivity (%) | 101 | |
| 95% specificity | | 73 |
| 98% specificity | | 62 |

As can be seen from Table 3, at a specificity of 95%, elevated values of TIMP-1 are detected in 73% of the CRC patient stool samples. At a specificity level of 98%, elevated values of TIMP-1 are detected still detected in 62% of the CRC patient stool samples.

### Example 5

### Combinations of TIMP-1 with Other Stool Markers

Combinations of TIMP-1 with other biomarkers from stool extracts were evaluated. The markers hemoglobin, the hetero-complex of hemoglobin with haptoglobin, Calprotectin, M2-PK and CEA are measured using commercial ELISAs. Assays for measurement of hemoglobin, hemoglobin/haptoglobin and calprotectin are obtained from R-Biopharm, Germany. The Calpro Calprotectin ELISA is manufactured by Calpro SA, Norway, and is marketed outside of Germany as PhiCal Test. The assay for measurement of CEA is obtained from Roche Diagnostics, Germany. The assay for M2-PK is supplied by Schebo Biotech, Germany. While some of the assays are intended for measurements in stool extracts, for CEA stool is not a commonly used sample material. Hence, the assays have to be adjusted to the measurement of the corresponding analyte in a sample representing an extracted stool specimen. The samples (stool extracts) are prediluted 20-fold for CEA determinations, but otherwise the assay is run according to the manufacturers recommendations.

TABLE 4
Overview of assays and extract dilutions used
for the measurement in stool extracts

| | | | Total extract |
|---|---|---|---|
| | | Stool Net Weight/ | dilution to |
| Assay | Supplier | Extraction method | assay |
| Hb | R-Biopharm | See example 2 | 1:10 |
| Hb-Haptoglobin | R-Biopharm | See example 2 | 1:10 |
| TIMP-1 | R&D-Systems | See example 2 | 1:6 |
| CEA | Roche-Elecsys | See example 2 | 1:400 |
| Calprotectin | Calpro AS | See example 2 | 1:51 |
| M2-PK | Schebo-Biotech | Original sample device | 1:6 |
| | | from Schebo Biotech | |

To test if a marker combination will improve the diagnosis of CRC, the markers are combined by Bayes Logistic Regression (BLR). In the BLR algorithm for the evaluation of marker combinations a Gaussian prior is used and implemented in the BBR-Software of Alexander Genkin, David D. Lewis, and David Madigan (Large-scale Bayesian logistic regression for text categorization. Technometrics). The following settings are used: no automatic feature selection, prior variance fixed at 0.05, no threshold-tuning, and input standardization by normalization. For the numerical process the default settings with a convergence threshold of 0.0005, 1000 iterations and no-accuracy-mode are retained unchanged. The results with the basic algorithm get evaluated by 100 runs in a Monte-Carlo cross-validation design. In each run, two-third of all cases and controls, respectively, are selected as training set via the MATLAB R2006a in-built function randsample with starting value 19022007 for the default random number generator. The basic algorithm is applied on the training set to generate a diagnostic rule. A threshold on the estimated posterior case-probabilities is determined on the controls of the training set to achieve a specificity of 95% or 98%, respectively. The diagnostic rule is then applied to the other third of the data to estimate sensitivity and specificity at the given threshold.

For a screening assay not only the ROC value is relevant. A quite critical requirement in a screening setting is a good enough sensitivity at a high specificity. High specificity is crucial because a low specificity would cause a high number of false positive results accompanied by unnecessary follow-up procedures and distress for the patients. Table 5 summarizes the ROC values of the evaluation of the 101 CRC patients versus the 252 controls as specified in Table 1 together with the sensitivities at a preset specificity of 95% and 98%, respectively.

TABLE 5
Marker combinations for the detection of CRC

| | Marker combination | | | | | |
|---|---|---|---|---|---|---|
| | | TIMP-1 + | TIMP-1 + | TIMP-1 + | TIMP-1 + | TIMP-1 + |
| | TIMP-1 | Hb-Hp | Hb | Calpro. | M2-PK | CEA |
| ROC area %) | 91 | 95 | 94 | 92 | 92 | 91 |
| Sensitivity at 95% | 73 | 88 | 85 | 79 | 79 | 73 |
| Spec. | | | | | | |
| Sensitivity at | 62 | 79 | 73 | 64 | 70 | 61 |
| 98% Spec. | | | | | | |

As can be deduced from Table 5, the AUC values for the individual markers in the diagnosis of CRC are very similar. On the other hand the sensitivity in detection of CRC can be significantly improved by combination of TIMP-1 with other markers of CRC. This is particularly evident at a specificity level of 98%. While TIMP-1 alone has a sensitivity of 61% at a specificity level of 98%, the combination with other CRC markers, like Hb, M2PK and Hb/Hp increases the sensitivity. Particularly the marker combination consisting of TIMP-1 and the hemoglobin-haptoglobin complex shows an increase in sensitivity from 62% to 79%. Marker combinations comprising TIMP-1 are therefore considered very important in order to detect CRC at early stages. Particularly such combination appears to be valuable in the detection of a colorectal cancer at stage I or II, respectively.

What is claimed is:

1. A method for diagnosis of colorectal cancer in an individual comprising the steps of:
a. providing a stool sample obtained from the individual,
b. contacting the sample with a specific binding agent for tissue inhibitor of metalloproteinase 1 (TIMP-1) under conditions appropriate for formation of a complex between the binding agent and TIMP-1,
c. determining an amount of complex formed, and
d. correlating the amount of complex formed to the diagnosis of colorectal cancer in the individual.

2. A method for diagnosing colorectal cancer in an individual comprising the steps of:
a. providing a stool sample obtained from the individual,
b. contacting the sample with a specific binding agent for tissue inhibitor of metalloproteinase 1 (TIMP-1) under conditions appropriate for formation of a complex between the binding agent for TIMP-1 and TIMP-1,
c. contacting the sample with a specific binding agent for a marker selected from the group consisting of hemoglobin/haptoglobin complex, hemoglobin, calprotectin, and tumor M2 pyruvate kinase (M2-PK) under conditions appropriate for formation of a complex between the binding agent for the marker and the marker,
d. detecting the amount of complex formed in steps (b) and (c), and
e. correlating the amount of complex formed in steps (b) and (c) to the diagnosis of colorectal cancer in the individual.

3. The method according to claim 2 wherein the marker is hemoglobin.

4. The method according to claim 2 wherein the marker is hemoglobin/haptoglobin complex.

5. The method according to claim 1 wherein the specific binding agent is an antibody.

6. The method according to claim 2 wherein the specific binding agent is an antibody.

7. The method according to claim 1 wherein following step (a) and prior to step (b), the stool sample is processed using an extraction buffer.

8. The method according to claim 2 wherein following step (a) and prior to step (b), the stool sample is processed using an extraction buffer.

9. The method according to claim 1 wherein in the step of providing a stool sample, a stool collection device is used that contains an extraction buffer.

10. The method according to claim 2 wherein in the step of providing a stool sample, a stool collection device is used that contains an extraction buffer.
